(19) United States
(12) Patent Application Publication
(10) Pub. No.: US 2005/0187598 A1
(43) Pub. Date: Aug. 25, 2005

(54) Mat

(76) Inventors: Eiko Shimizu, Gunma (JP); Kenkichi Kuwabara, Gunma (JP)

Correspondence Address: BURNS DOANE SWECKER & MATHIS L L P, POST OFFICE BOX 1404, ALEXANDRIA, VA, 22313-1404, US

(21) Appl. No.: 10/516,351

(22) PCT Filed: Jun. 25, 2003
(86) PCT No.: PCT/JP03/08035

(30) Foreign Application Priority Data
Jun. 26, 2002 (JP) 2002-185476

(51) Int. Cl. A61F 7/00

(52) U.S. Cl. 607/96

(57) ABSTRACT

The present invention provides a mat (1, 4) with multiple filing chambers (10a, 40a) charged gel-liked heat medium (12) and expanded by a pressure of the heat medium, wherein the filling chambers in parallel and adjacent to each other. The mat (1, 4) of the invention can keep simply and effectively a whole-body or a part of a patient warm or cool and can also prevent from bed sore.

### TECHNICAL FIELD

The present invention relates to a mat which prevents bed sore and also keeps the body warm or cool.

### BACKGROUND ART

When the body is expected to be kept in the same posture in surgical operation, medical care or the like, a measure for preventing bed sore caused by interruption in blood circulation accompanied by compression of capillaries is required.

In such a bed sore, capillaries are compressed by a patient's own weight to cause interruption in blood circulation, whereby rubefaction of the skin starts, and the condition gradually becomes serious.

In order to prevent the interruption in blood circulation by this compression, for example, a decompression tool such as an air mat, a resinous mat or a beads-filled pad has been hitherto used so as not to concentrate a pressure on a contact site between the human body and a mat or the like.

However, for example, since an air mat is quite resilient, it has been problematic in stability of an operation site during surgical operation. Further, it has been excessively collapsed to give no satisfactory effect of preventing bed sore, and have involved problems in size, maintenance, noise of an air pump and the like.

The resinous mat includes those using a urethane foam and a dry polymer (trade name: ACTON DRY POLYMER).

Of these resinous mats, a certain effect of dispersing a contact pressure might be expected, but prevention of bed sore is not satisfactory.

In addition, there is, for example, a beads-filled pad in which expanded resin beads are accommodated in a woven fabric enriched in stretchability or the like. However, this has been, like the resinous mats, also unsatisfactory in preventing bed sore.

Further, mere rubefaction accompanied by interruption in blood circulation is, in some conditions such as a patient's state and an operation time, developed into more serious bed sore during surgical operation or in perioperative absolute rest which has quite an adverse effect on recovery of a patient.

In order to avoid this situation, observation of a site in which occurrence of bed sore is expected and a change of a posture are quite important.

However, even during surgical operation or before or after surgical operation, the observation of the site or the change of the posture is sometimes impossible in some state of a patient. In this case, it has been impossible to prevent bed sore.

Even though the observation of the site or the change of the posture has been possible, it has been required to conduct the same by doctors, male nurses or nursing-care persons. Thus, there has been a problem that a load of nurses and the like is great.

In case of a patient suffering from disturbance of consciousness or disorder of moving function, bed sore could not have been prevented sufficiently even with an ordinary decompression tool. Accordingly, there has been a problem that nursing-care persons and the like have to often change the posture which invites a great load.

As a mat from which the effect of preventing bed sore is expected, there is a waterbed.

However, since a patient's body is sunk too much in the waterbed, occurrence of bed sore cannot be prevented satisfactorily.

The waterbed has been problematic in that it is itself large-scaled, quite heavy, hard to perform installation, movement or the like, costly, and unrealistic when used in hospitals, in nursing-care institution or at home.

A problem to which attention has to be paid during surgical operation is to prevent shivering accompanied by abrupt decrease in body temperature.

The temperature inside the human body is from approximately 38 to 40° C., though it varies with the age, the sex, the condition of diseases and the like. For example, when surgical operation of abdominal section is performed in an operating room at room temperature of approximately 25° C., a body temperature of a patient is decreased at once to cause shivering.

In surgical operation, it is therefore necessary to control the body temperature. Warming has been hitherto conducted using a warming tool such as a warm air heater, a warm air mat or a warm water circulation-type blanket lest the patient's body temperature should be decreased.

Nevertheless, such an ordinary method has involved problems that a device such as a heater has to be put near an operating table, it is obstructive and noisy, and the device itself is liable to get out of order.

When these warming tools are, like a warm water circulation blanket, laid under a patient, they have to be inserted between a decompression tool and a patient upon taking preference for keeping warm. In this case, the decompression effect is notably decreased.

On the contrary, upon taking preference for decompression, the decompression tool has to be put between the warming tool and the patient, and the effect of keeping the body temperature is decreased.

Thus, keeping the body temperature in surgical operation is contradictory to prevention of bed sore.

As a device having both the function of keeping the body temperature and the function of preventing bed sore, there is a resinous mat having a cord heater embedded therein.

However, there have been problems that the resinous mat itself does not have a satisfactory function of preventing bed sore and the cord heater tends to get out of order owing to disconnection or the like.

With the advancement of the aging society, the number of bedridden persons is increased, and prevention of bed sore is a serious problem in nursing care in institution or at home. However, there is no simple and effective mat that can solve this problem. At present, there has been no choice but to rely on manual labor of nursing-care persons and the like, inviting a problem of a great burden.

When a whole body or a predetermined site of a lying patient has to be cooled, a cooling tool such as an ice pack is sometimes inserted between the patient and a bed or the like.

In this case, the cooling tool is closely adhered to a region in which capillaries are compressed by the weight of the patient to interrupt blood circulation. Thus, it has involved problems that frostbite might occur and interruption of blood circulation is accelerated to prompt bed sore.

For this reason, when cooling is conducted using an ordinary cooling tool such as an ice pack, a contact portion of the cooling tool and a condition of blood circulation and a cooling state in the contact region have to be confirmed at any time. Thus, there has been a problem that it takes much labor to control the conditions.

Accordingly, the invention aims to provide a mat which can prevent bed sore and keep a whole body or a part of a patient warm or cool simply and effectively.

### DISCLOSURE OF THE INVENTION

The present inventors have assiduously conducted investigations to solve the foregoing problems, and have consequently found that it is possible to prevent bed sore and keep a whole body or a part of a patient warm or cool simply and efficiently using a mat in which multiple filling chambers formed of flexible sheets and arranged such that they are in parallel and adjacent to each other are provided, a gel-like heat medium is charged into the filling chambers, and the filling chambers are expanded by a pressure of the heat medium. This finding has led to the completion of the invention.

That is, the invention is specified by the following (1) to (8).

(1) A mat comprising multiple filling chambers which are in parallel and adjacent to each other, wherein the filling chambers are formed of flexible sheets, a gel-like heat medium is charged into the filling chambers, and the filling chambers are expanded by a pressure of the heat medium.

(2) The mat recited in (1), wherein the heat medium is charged into the filling chamber by charging the heat medium into a bag capable of being inserted closely into the filling chamber, inserting the bag having the heat medium charged therein into the filling chamber and sealing the filling chamber.

(3) The mat recited in (1) or (2), wherein an emboss formed by bonding predetermined regions of wall surfaces forming the filling chamber and opposite to each other is provided in the vicinity of at least one end of the filling chamber.

(4) The mat recited in (1) or (2), wherein the sheets forming the filling chamber are a pair of opposite thermoplastic resin sheets which are heat-bonded along the contours of the filling chamber to define the filling chamber.

(5) The mat recited in (1) or (2), wherein flexible fins are provided which are extended outwardly from at least a part of margins.

(6) The mat recited in (1) or (2), wherein the heat medium is a warming material.

(7) The mat recited in (1) or (2), wherein the heat medium is a cooling material.

(8) The mat recited in (1) or (2), wherein the heat medium is a hydrogel.

### BRIEF DESCRIPTION OF THE DRAWINGS

FIG. 1 is a top view showing a first working example of a mat according to the invention.

FIG. 2 is a sectional end view taken on line A-A shown in FIG. 1.

FIG. 3 is a partial explanatory view showing an example of a use state of the mat shown in FIG. 1.

FIG. 4 is a top view showing a second working example of the mat according to the invention.

### BEST MODE FOR CARRYING OUT THE INVENTION

The invention is described in detail below by referring to drawings.

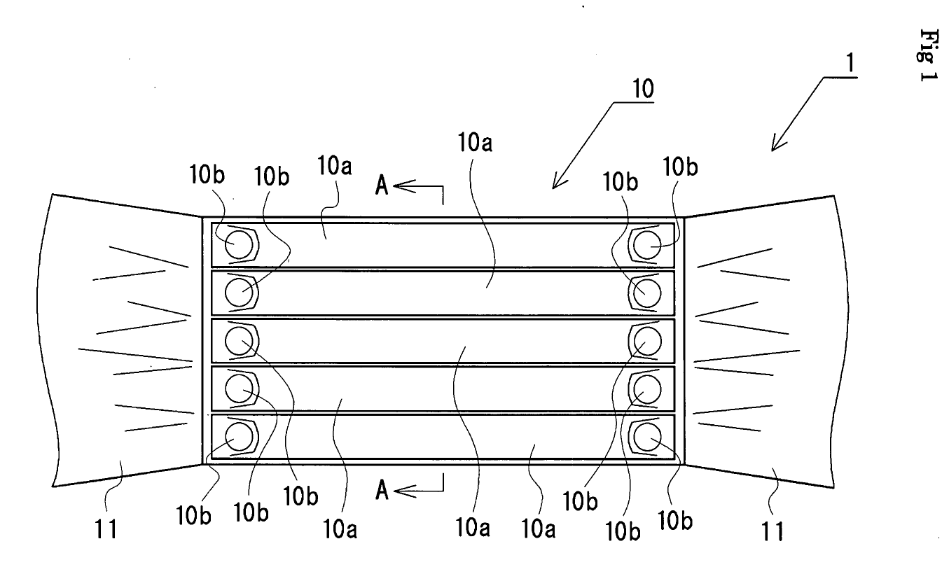

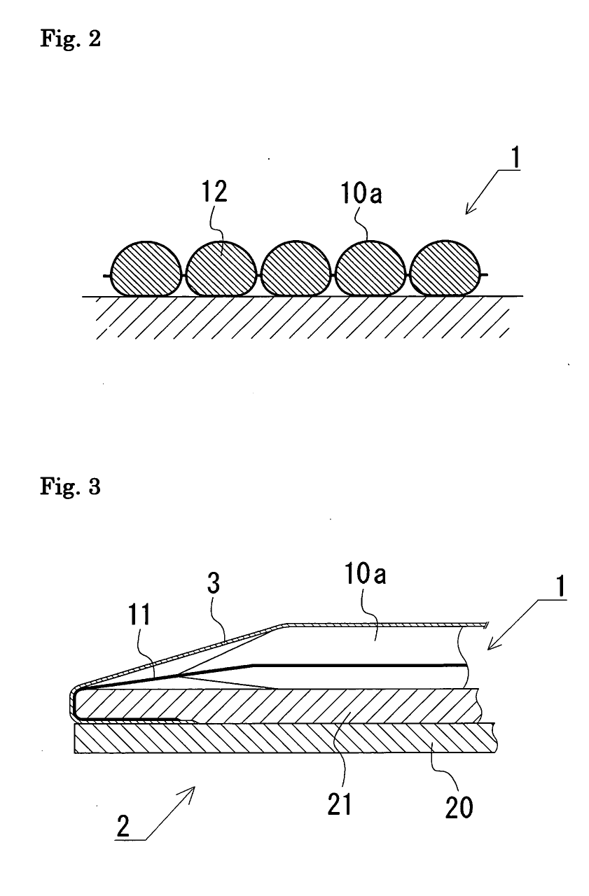

To begin with, the first working example of the mat according to the invention is described on the basis of FIG. 1 and FIG. 2.

A mat 1 comprises a mat portion 10, fins 11 and a heat medium 12.

The mat portion 10 is formed by overlaying a pair of rectangular sheets made of a thermoplastic resin and bonding both of the sheets along the contours of a filling chamber 10a through melt-bonding with a high-frequency welder or the like.

Five such filling chambers 10a are defined in a long rectangular shape along a longitudinal direction of the sheets such that they are in parallel and adjacent to each other.

In the filling chambers 10a, the heat medium 12 is filled the process of the definement of the filling chambers 10a, and sealed within the filling chambers 10a.

As a filler 12, a hydrophilic polymer containing a large amount of water, namely a hydrogel, is used.

In order to increase a pressure of the heat medium 12 within the filling chambers 10a, regions opposite to each other in the sheets of wall surfaces forming the filling chambers 10a are heat-bonded circularly to form embosses 10b in the vicinity of both ends of the respective filling chambers 10a. Consequently, the filling chambers 10a are strongly expanded by the pressure thereof as shown in FIG. 2.

The fins 11 are rectangular thermoplastic resin sheets which have the same width as opposite edges on end sides of the filling chambers 10a in the mat portion 10 and which are heat-bonded to the mat portion 10 so as to be extended outwardly from the opposite ends.

When the mat 1 is used, the mat 1 is first stored in a warming box not shown, and warmed satisfactorily until the heat medium 12 reaches a predetermined temperature.

This warming box is adapted to be warmed at an appropriate temperature of less than 50° C., and the mat 1 stored therein is warmed by radiation heat or conduction heat.

Since the temperature of the surface of human body is generally from 32 to 33° C., it is advisable that the warming box can warm the surface of the mat 1 at from approximately 34 to 36° C.

Next, the mat 1 warmed satisfactorily is withdrawn, and installed in a desired position on a mattress 21 of an operating table 2 such that the fins 11 are hanged from both sides of the operating table 2.

The fins 11 hanged on both sides of the operating table 2 are then rolled, as shown in FIG. 3, under the mattress 21, and inserted into gaps between a base 20 and the mattress 21. Further, it is covered with a sheet 3, and the margins of the sheet are also rolled under the mattress 21, and inserted similarly.

When a patient lies on the operating table 2 with the mat 1 installed in this manner, the body of the patient is held by the multiple filling chambers 10a expanded in the shape of a nearly round rod.

At this time, the filling chambers 10a support the body of the patient with an appropriate hardness without concentrating a load on the contact surface with the mat 1. Thus, compression of capillaries does not occur.

Since the patient is kept warm with the warmed mat 1 to stimulate blood circulation, the bed sore does not occur.

When the abdominal operation is performed, the body temperature is not rapidly decreased owing to the warming effect of the mat 1, so that shivering can be prevented.

At the time of using the mat 1, the mat 1 allows insulation with the body of the patient or the cover put on the patient, so that the warming effect is maintained for a long period of time.

Accordingly, in surgical operation over a long period of time, occurrence of bed sore or shivering can be prevented.

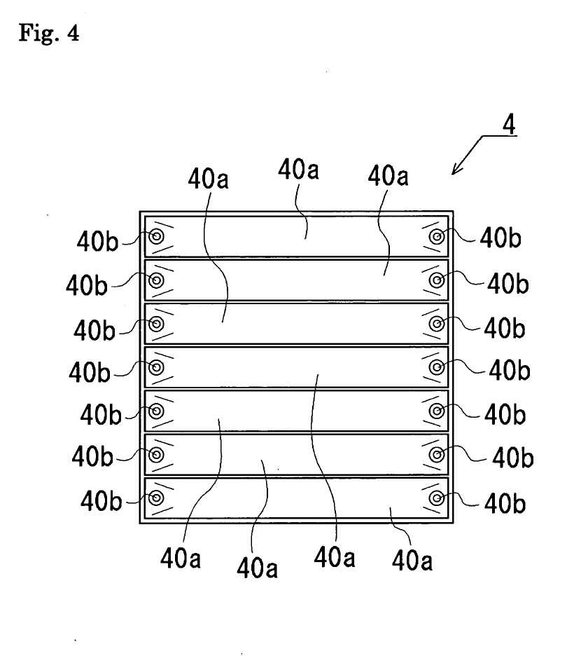

Next, a second working example of the mat according to the invention is described on the basis of FIG. 4.

By the way, since the main construction of this working example is the same as that of the first working example, different points thereof are mainly described.

A mat 4 has seven filling chambers 40a in which a heat medium has been charged.

These filling chambers 40a are strongly expanded by a pressure thereof which is increased with embosses 40b.

This mat 4 is appropriate as a pad of a cushion for a wheelchair.

In a wheelchair on which the cushion for a wheelchair having this mat 4 accommodated therein has been installed, rubefaction or bed sore does not occur on the skin of a wheelchair user in contact with the cushion even though the user sits thereon over a long period of time.

In this case, the cushion may be used while being warmed at an appropriate temperature by warming the mat 4 before use or mounting a heater in the cushion itself.

It is recommendable that an insulation layer such as an urethane mat is disposed between the mat 4 and the sitting surface of the wheelchair so as to accumulate the body temperature of the user in the mat 4 efficiently.

This working example is constructed as mentioned above. An appropriate material of the sheets and the fins constituting the mat is an ethylene film in view of a processability, a strength and the like. However, this is not critical.

Different materials may be used in the sheets constituting the mat portion and the fins, and different materials may also be used in the pair of sheets constituting the mat portion.

Specific examples of the heat medium include poly(N-vinylacetamide), polyvinyl alcohol, polyethylene oxide, polyvinyl pyrrolidone, carrageenan, alginic acid, sodium carboxymethylcellulose, carboxymethyl starch sodium salt, polyacrylic acid, sodium polyacrylate, polyacrylamide, agar, polyaspartic acid, sodium polyglutamate, poly(ε-lysine) and the like. However, these are not critical, and plural hydrogels may be incorporated.

As the hydrogel, a hydrogel obtained by mixing a hydrophilic polymer such as polyvinyl alcohol, polyethylene oxide or polyvinyl pyrrolidone with water and applying radioactive rays to the mixture to cause a crosslinking reaction may be used.

With respect to the size of the mat according to the invention, in case of, for example, a mat having five filling chambers as shown in FIG. 1, the length of the filling chamber is 500 mm, the width over all of the filling chambers is 300 mm, the diameter of the emboss is 20 mm, and the height near the center of the filling chamber expanded when put on a flat surface is 30 mm, so that the weight is approximately 3 kg. This mat is then easy to use.

The number of the filling chambers is not limited to five, and it may be four or less, or six or more according to the use purpose.

For example, in case of a mat having four filling chambers, the length of the filling chamber is 500 mm, the width over all of the filling chambers is 238 mm, the diameter of the emboss is 20 mm, and the height near the center of the filling chamber expanded when put on a flat surface is 25 mm, so that the weight is approximately 2 kg. This mat is then easy to use.

The size of each portion constituting the mat and the number of the filling chambers vary with the use purpose and the use conditions, and the foregoing sizes are naturally not critical. They can properly be changed within the range of the invention. However, when the mat is large-sized or the number of the filling chambers is increased, the weight and the bulk are increased, and the mat is hard to handle.

Accordingly, when the mat is put over a wide area, it is recommendable that a combination of plural mats having the foregoing sizes is used by being set on positions on which the head, shoulders, arms, buttocks, legs, heels and the like are placed.

The mat according to the invention has the greatest characteristic feature that the body can be held by using, as a cushion, the filling chambers expanded by the pressure of the gel-like heat medium, and the construction of the details is not limited to the forgoing working examples. For example, with respect to the sheet constituting the mat portion and the fins, any sheet will do so long as it has a flexibility, a water resistance and a strength that withstands the use and can be processed into a desired shape. For example, it may be a fiber-reinforced sheet which is reinforced with a nonwoven fabric or a woven fabric or a composite material obtained by laminating plural materials.

A method for defining the filling chambers is not limited to the foregoing working examples. For example, the filling chambers maybe formed by connecting the filling chamber portions formed in the shape of a round rod in parallel or by bonding the surface sheet in a wavy state to the flat reverse sheet.

The embosses provided to increase the pressure of the heat medium filled in the filling chambers are not necessarily provided so long as the pressure of the heat medium can fully be secured by filling the heat medium. The number, the shape and the area of the embosses can freely be changed so long as the embosses can expand the filling chambers satisfactorily.

The heat medium is not directly charged into the filling chamber but may be charged into the filling chamber by, for example, previously charging the heat medium into a long bag having approximately the same shape as that of the filling chamber, inserting the bag into the filling chamber and sealing the filling chamber. In this case, the work of charging the heat medium becomes easy, and leak of the heat medium due to breakage of the filling chamber can be prevented.

This bag may have any shape so long as it can closely be inserted into the filling chamber, and plural bags may be inserted into one filling chamber.

A method for fixing the mat on an operating table, a bed or the like is not limited to using the fins. For example, a surface fastener which can freely be attached to or detached from the sheet or the mattress may be put on the reverse surface of the mat, or plural fixing methods may be combined.

A fin-free mat may be put into a bag, a cover or the like, and fastened on the operating table. A desired stopper may be provided on the mat for fixing.

Plural mats may be installed on a mat holder capable of installing plural mats, and placed on a bed, so that the mats are not slid by the weight of the mats during use.

Any method for installing the mat on a bed or the like is available so long as the mat is not slid during use.

For some use purpose, an installation unit such as fins is not necessarily provided.

In the foregoing working examples, the hydrogel is used as the heat medium. Accordingly, this mat can be cooled in a cooling chamber or the like and used as a cooling mat.

As the heat medium, any gel-like heat medium may be used.

As this heat medium, a heat medium for keeping warm and cool is generally used. A heat medium for keeping warm only or for keeping cool only is also available.

When the mat according to the invention is used for cooling, interruption in blood circulation due to compression does not occur in capillaries around an affected part, with the result that the affected part can be cooled properly.

In the working examples, the mat is used by being fixed on the operating table. The mat is used not only during surgical operation, but also in a usual bed or mattress. Further, the mat can be used in any environment such as an institution of medical care, an institution of nursing care and home. The posture of a user includes lying with his face upward, lying with his side, lying with his face downward and the like. A special posture according to the necessity of medical treatment or medical care is also available.

The field of using the mat according to the invention is not limited to medical treatment, welfare and nursing care. For example, the mat may be utilized in a kneeling cushion, a cushion, a pillow and various pads by changing the length and the width of the mat and the number of the filling chambers.

When the mat according to the invention is used as a cushion of a chair for office work, a person can continue work efficiently without feeling pain in the buttocks even though sitting for a long period of time.

The mat according to the invention may be used without being warmed. At this time, the body temperature of a user is accumulated in the heat medium charged in the filling chambers, and the mat exhibits the same effect of keeping warm as the mat previously warmed during use.

### EXAMPLES

The invention is illustrated more specifically by referring to Examples. However, these Examples are mere working examples, and do not limit the invention. They may be changed without departing from the scope of the invention.

The test was conducted using the same mat as shown in FIG. 1.

With respect to the size of the mat used, the length of the filling chamber was 500 mm, the width over all of the filling chambers was 300 mm, the diameter of the emboss was 20 mm, and the height near the center of the filling chamber expanded when put on a flat surface was 30 mm.

The mats were warmed in a warming box for a predetermined period of time. The warmed mats were fastened in parallel on a hard top plate, and a sheet was put thereon.

At this time, the surface temperature of the mats was 34° C.

The inventor lay on the mats through the sheet with his face upward, and was covered with a blanket from feet to shoulders.

He kept a rest with this posture. After the lapse of 10 hours, the condition of the skin around the blade bone, the sacred bone and the heels was observed. Consequently, rubefaction did not occur in any of these positions.

The surface temperature of the mats was 33° C., and this temperature was almost unchanged.

The results revealed that the use of the mats could prevent bed sore and maintain the effect of keeping warm for a long period of time.

### INDUSTRIAL APPLICABILITY

The mat can prevent bed sore and keep a whole body or a part of a patient warm or cool simply and effectively.

When the mat is used as a measure for preventing the decrease in body temperature of a patient in surgical operation, there is no need to bring extra devices or the like into an operating room, so that surgical operation can proceed smoothly.

When there is a warming box or a refrigerator, the mat can easily be used not only in an institution of medical treatment and an institution of nursing care, but also at home to decrease a load of nursing-care persons and the like.

1. A mat comprising multiple filling chambers which are in parallel and adjacent to each other, wherein the filling chambers are formed of flexible sheets, a gel-like heat medium is charged into the filling chambers, and the filling chambers are expanded by a pressure of the heat medium.

2. The mat as claimed in claim 1, wherein the heat medium is charged into the filling chamber by charging the heat medium into a bag capable of being inserted closely into the filling chamber, inserting the bag having the heat medium charged therein into the filling chamber and sealing the filling chamber.

3. The mat as claimed in claim 2, wherein an emboss formed by bonding predetermined regions of wall surfaces forming the filling chamber and opposite to each other is provided in the vicinity of at least one end of the filling chamber.

4. The mat as claimed in claim 2, wherein the sheets forming the filling chamber are a pair of opposite thermoplastic resin sheets which are heat-bonded along the contours of the filling chamber to define the filling chamber.

5. The mat as claimed in claim 2, wherein flexible fins are provided which are extended outwardly from at least a part of margins.

6. The mat as claimed in claim 2, wherein the heat medium is a warming material.

7. The mat as claimed in claim 2, wherein the heat medium is a cooling material.

8. The mat as claimed in claim 2, wherein the heat medium is a hydrogel.

9. The mat as claimed in claim 1, wherein an emboss formed by bonding predetermined regions of wall surfaces forming the filling chamber and opposite to each other is provided in the vicinity of at least one end of the filling chamber.

10. The mat as claimed in claim 1, wherein the sheets forming the filling chamber are a pair of opposite thermoplastic resin sheets which are heat-bonded along the contours of the filling chamber to define the filling chamber.

11. The mat as claimed in claim 1, wherein flexible fins are provided which are extended outwardly from at least a part of margins.

12. The mat as claimed in claim 1, wherein the heat medium is a warming material.

13. The mat as claimed in claim 1, wherein the heat medium is a cooling material.

14. The mat as claimed in claim 1, wherein the heat medium is a hydrogel.
